(19) United States
(12) Patent Application Publication
(10) Pub. No.: US 2009/0036795 A1
(43) Pub. Date: Feb. 5, 2009

(54) SUBSTANCE SAMPLING AND/OR SUBSTANCE DELIVERY VIA SKIN

(75) Inventors: Paulus Cornelis Duineveld, Drachten (FR); Fokke Roelof Voorhorst, Drachten (FR); Michel Allan Aurelius Schallig, Drachten (FR); Anke Sinnema, Drachten (FR)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/067,947

(22) PCT Filed: Sep. 22, 2006
(86) PCT No.: PCT/IB2006/053435

§ 371 Date: Aug. 22, 2008

(30) Foreign Application Priority Data
Sep. 26, 2005 (EP) 05300773.8

(51) Int. Cl. A61B 5/00; A61B 10/00; A61B 17/00

(52) U.S. Cl. 600/570; 600/573; 604/22

(57) ABSTRACT

In a technique for substance sampling and/or substance delivery via the skin barrier, the skin is domed and then a portion removed. Typically a contact portion of the apparatus effects doming of the skin and a cutting, grazing or scraping arrangement removes a portion of the domed skin. The doming of the skin and hence the skin removal is closely controlled. Samples may be taken via the removed portion of the skin and/or substances delivered therethrough.

The present invention relates to substance sampling and/or substance delivery via the skin, particularly in healthcare applications

Measuring the concentration of special molecules in the interstitial body fluid (e.g. glucose) can provide information about the physical condition of a patient. Drug delivery through the skin, i.e. transdermal drug delivery, is an emerging method for controlled and/or continuous delivery of drugs. Transdermal drug delivery has a big advantage over oral delivery because with the latter, macromolecules are either broken down in the gastro-intestinal tract or are too large to be absorbed across the gastro-intestinal epithelium. In the pharmaceutical industry a growing number of medicines are based on macromolecules to be used in, such as gene therapy, enzyme replacement and disease immunization. On the other hand, certain medical diseases such as diabetes, also have a need for continuous, reliable and painless sampling and injection of fluids.

Normally the skin outer layer, i.e. the stratum corneum, is a very effective barrier for components travelling in and out of the body. Frequently utilised techniques for transdermal delivery of drugs and withdrawal of interstitial fluid (or blood) is by needles. This is, however, a painful method, as the needle is penetrating the skin deep enough to touch the nerves. Further, there is a relatively high risk of infection and, after multiple usage at the same position, the risk of thromboses. Also, a needle is a less suitable method for very frequent or even continuous use. Therefore there is a desire for a more pain free, and reliable, method for sampling and delivery of fluids.

In the last decade several techniques have been developed for transdermal sampling and injection. These techniques are discussed with their advantages and disadvantages, in S. E. Cross and M. S. Roberts, ‘Physical enhancement of transdermal drug application: is delivery technology keeping up with pharmaceutical development?’ Current drug Delivery, Vol 1, 81-92, 2004. Several known techniques are electrically based: iontophoreses (where a small electrical current is passed through the skin), electroporation (where a short electrical pulse is used), ultrasound, laser ablation and laser generated stress waves. On the other hand there are also structure-based techniques such as microneedles, where an array of very small needles is punched through the skin. These needles are very small, of order 100 μm and do not penetrate deep enough to cause pain. Finally there are also velocity-based techniques, such as jet injection, where fluid droplets at high velocity (larger than 60-100 m/s) are passing through the skin.

Micro-needles are however fragile, susceptible to breakage and need to be applied with care. Further, micro-needles punch the skin and the piece of skin that is punched has a risk of blocking the opening of the needle through which either the interstitial fluid (sampling) or drug (injection) has to pass, or the drug needs not to be in front of the opening in the needle where one wants to apply liquid. For continuous use over periods of months to a year, micro-needles may be less suitable as the body is likely to react to the needles.

An improved technique has been devised.

According to a first aspect, the present invention provides apparatus comprising means for doming and removing skin enabling substance sampling and/or substance delivery via the skin barrier.

The apparatus preferably comprises

contact means for contacting the skin, and including doming means for doming the skin; and

a skin removal arrangement arranged to pass adjacent and remove a layer of domed skin adjacent the doming means.

Doming of the skin should be understood as localised deformation to produce a dome or ridge of skin of controlled size. Doming of the skin is intended to cover like deformations, including pinching or rucking of the skin. Doming should be understood as preferential doming in a positive or upward sense, outwardly from the general surface of the skin, rather than negative doming by means of forming a depression in the skin. This will become readily apparent from the following description of the invention.

The contact means preferably includes an aperture for doming the skin, such that domed skin protrudes through the aperture, the cutting arrangement arranged to pass across the aperture of the contact means and remove domed skin adjacent the aperture.

The apparatus can be used for one or other of sampling or delivery. In certain embodiments the apparatus may be used for both sampling and delivery.

According to a second aspect, the present invention provides a method for sampling and/or substance delivery via the skin barrier, the method comprising doming the skin in a controlled manner and operating a skin removal arrangement to remove domed skin so forming an opening in the kin barrier; and either sampling interstitial fluid or other substance obtained via the opening, and/or delivering a substance from externally of the skin barrier through the removal zone.

The aperture of the contacting means preferably has an inclined sloping wedge like edge aiding in doming of the skin in the aperture. The wedge shaped edge of the aperture extends from a relatively wider distal open portion to a relatively narrower proximal open portion. A blade typically passes adjacent the narrower proximal open portion. Typically the apparatus includes a skin contacting lamellae arrangement, the lamella have a wedge shaped termination at the aperture, defining an inclined surface which aids in doming of the skin. The aperture may typically be circular, but other shaped apertures such as rectangular or square or elongate slits or slots may be utilised.

The technique relies on the skin being domed accurately (for example between two lamellae) and a blade (or other skin removal arrangement) removing the skin at a controlled plane. This technique enables very small openings to be accurately and repeatably made in the skin barrier by doming the skin. The technique is akin to certain known hair cutting (shaving) technology in which the skin is domed and the hair cut accurately without damaging or irritating the skin.

The skin is domed between two or more lamellae and the skin opening is very well made by cutting, typically with a blade, in a plane which is parallel to the non-deformed skin. This is a big difference with prior art techniques such as the method of making an opening by micro-needles, where moving a needle in a direction perpendicular to the non-deformed skin makes the opening. As the doming can be very well controlled, due to the well controlled thickness of the lamellae and shape and size of the opening, as well as the movement of the blade it is possible to make very small openings without hitting the nerves, this makes the method effectively painless. Further it is also possible to control the thickness of the opening, as well as the width of the opening by changing the opening diameter and doming of the skin.

Another advantage of the method is that the skin that is cut off is not blocking the opening as it can be removed by the blade away from the opening, due to the direction of movement of the blade.

The invention will now be further described by way of example only and with reference to the accompanying drawings, in which;

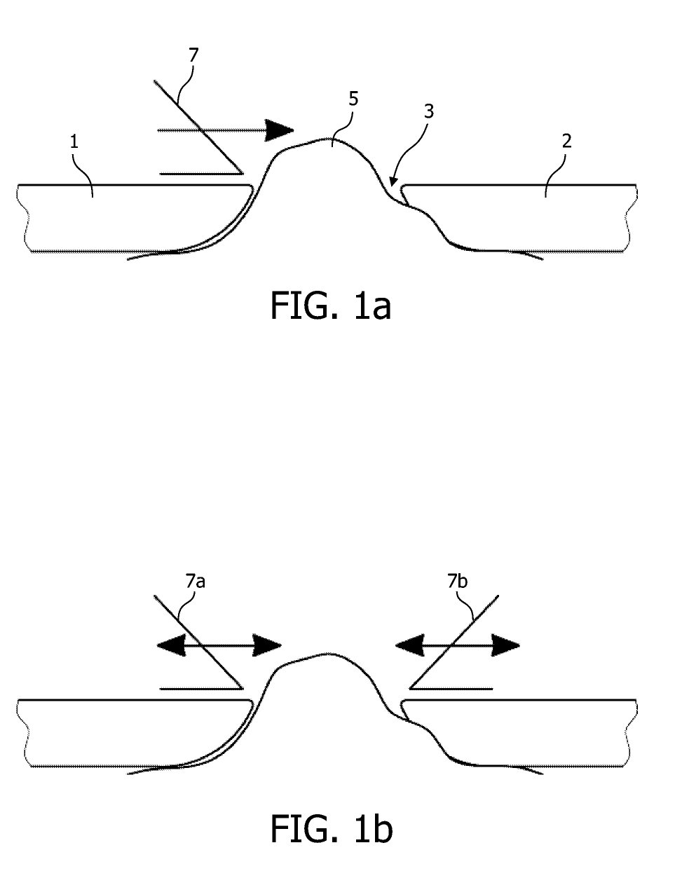

FIGS. 1a and 1b are schematic representations of a technique according to the invention;

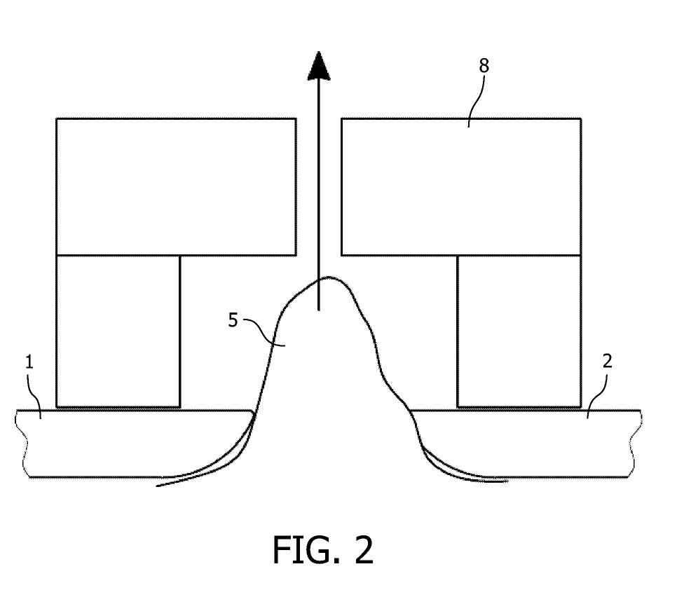

FIG. 2 is a schematic representation of a modified technique;

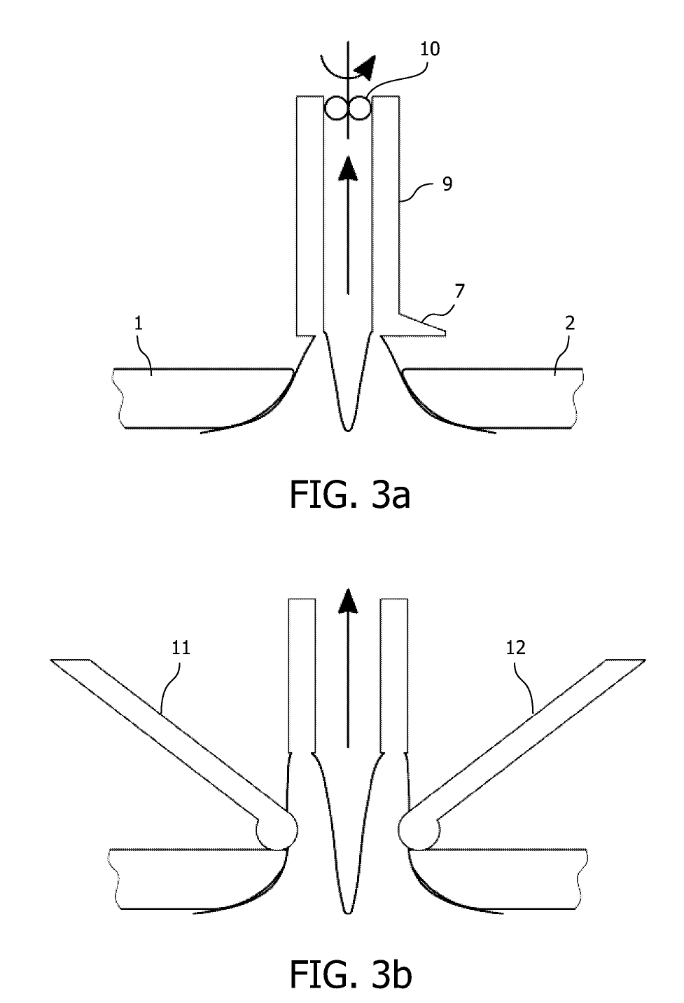

FIGS. 3a and 3b are schematic representations showing further modifications to the technique;

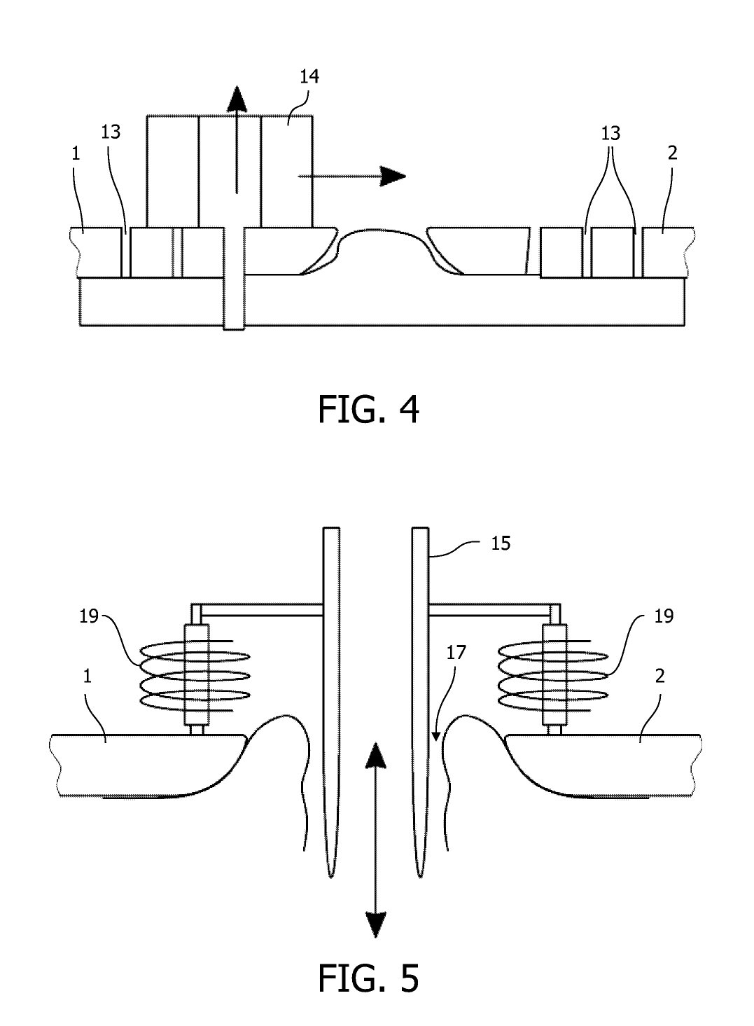

FIG. 4 is a schematic representation showing a further modified technique;

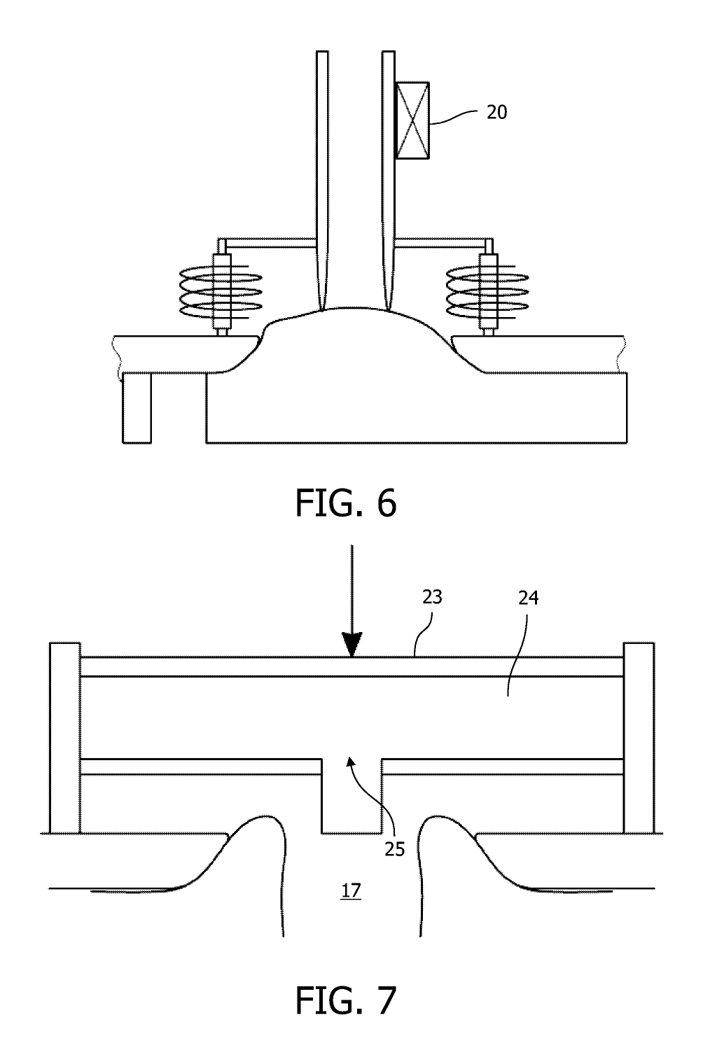

FIGS. 5 and 6 are schematic representations of a further modified embodiment of the invention;

FIG. 7 is a schematic representation of a further modified embodiment of the invention;

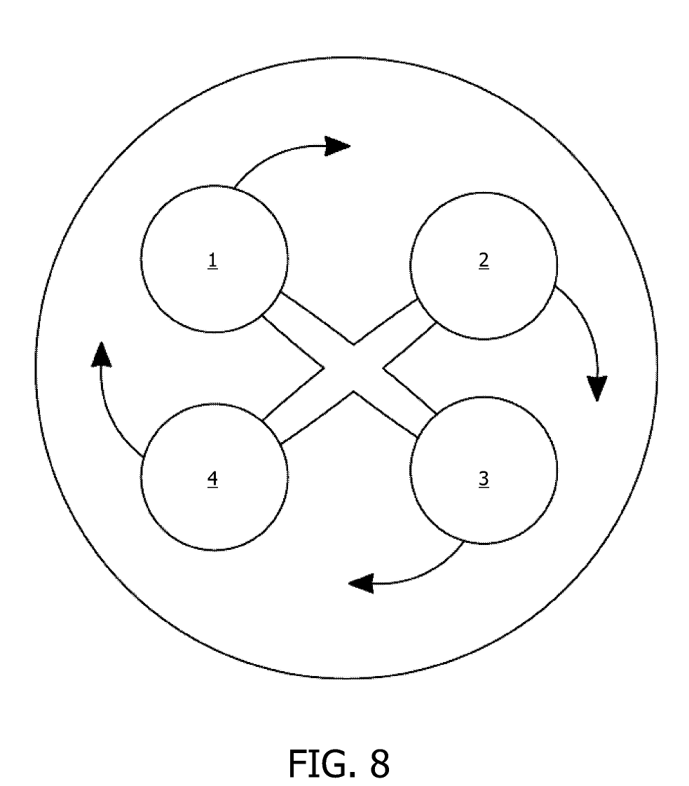

FIG. 8 is a schematic representation of a further modified embodiment in the form of a rotary carousel;

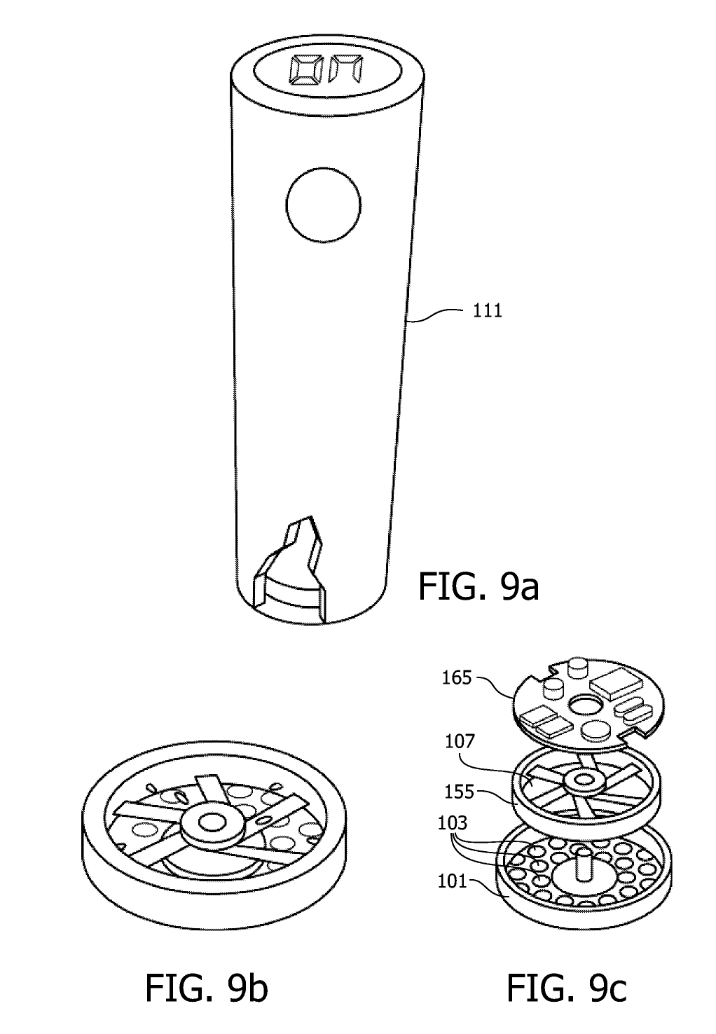

FIGS. 9a to 9c are schematic representations showing a further alternative embodiment in accordance with the invention; and

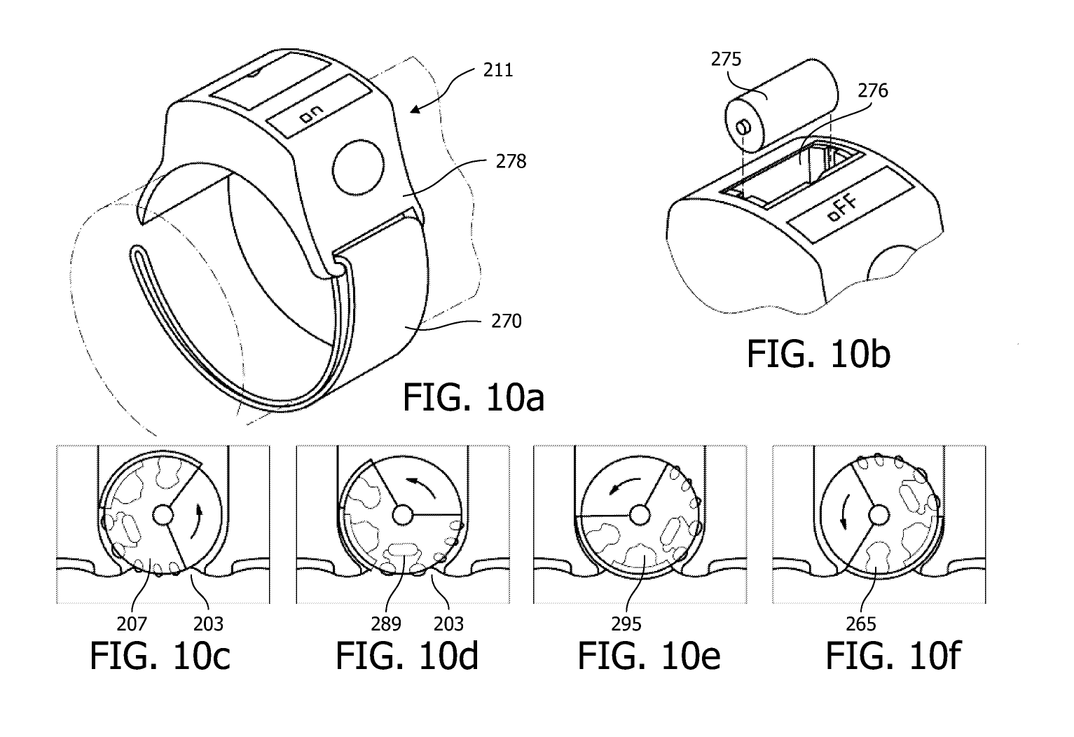

FIGS. 10a to 10f are schematic representations showing a further alternative embodiment in accordance with the invention.

Referring to the drawings, and initially FIGS. 1a and 1b, there is shown apparatus comprising a skin contacting probe having an end defined by lamella 1, 2 defining an aperture 3 there between. The lamella has a wedge shaped termination at the aperture 3 defining an inclined surface 4 which aids in doming of the skin 5 as will be described. A blade 7 is arranged to pass across the aperture in a slice plane that is closely adjacent the lamella 1,2.

The aperture 3 defined between the lamellae 1,2 may typically be a circular shape, but also rectangular or other shapes as a square or elliptical shapes are possible. The blade 7 which is used to make the opening in the skin may be rotating, as is drawn in FIG. 1a, or it may also be a multiple number of rotating blades. Further the skin may also be cut by a combination of two vibrating/reciprocating blades 7a,7b, as shown in FIG. 1b. The opening in the skin can be made in a single stroke of the blade, but may also be made in a series of contacts with the blade, where at every contact a small layer of the skin is removed. By placing multiple holes in a lamella it is possible to make a series of holes in the skin. The opening in the skin barrier may also be made by means of grinding, grazing, scraping or other like techniques. A clean cut by means of a blade is however envisaged as a preferred technique.

The main difference of this method with respect to other structural methods is that the skin is domed between an aperture in a lamella or between two or more lamellae and the skin opening is very well made by cutting with a blade that moves in a plane which is parallel to the non-deformed skin. This is a big difference with the method of making an opening by microneedles, where moving a needle in a direction perpendicular to the non-deformed skin makes the opening.

As the doming can be very well controlled, due to the well controlled thickness of the lamella and shape of the opening, as well as the movement of the blade it is possible to make very small opening without hitting the nerves, this makes the method instantly painless. Further it is also possible to control the thickness of the opening, as well as the width of the opening by changing the opening diameter and doming of the skin. Another advantage of the method is that the skin that is cut off is not blocking the opening as it can be removed by the blade away from the opening, due to the direction of movement of the blade.

Due to the direct contact between skin and lamella the method offers a very good seal. By applying a vacuum using vacuum apparatus 8 (as shown in FIG. 2) the doming of the skin can be enhanced. Conversely, by applying a positive pressure skin doming can be decreased. The used of an applied vacuum, or applied positive fluid pressure, has an effect that the nerves can be stimulated before cutting of the skin is done. This tends to overload the nerves with signals before the actual cutting is done. This may be an extra safety step for amelioration of pain.

When an opening in the skin is made by the technique of the present invention it is possible to extract interstitial fluid out of the skin for instance to use a conduit or tube 9 with a hollow opening, which is placed on top of the hole, as is sketched in FIGS. 3a and 3b. In the arrangement of 3a the tube 9 is mounted to the trailing end of the blade 7. Interstitial fluid will flow into the opening by capillarity or by applying a vacuum at the end of the tube. The vacuum can be applied by e.g. rotating a very small fan 10 as shown in FIG. 3a. The advantage of applying a vacuum is that the hole in the skin is very well located with respect to the opening between the lamella 1,2. Another method is forcing the interstitial liquid out of the body by applying a pressure on the skin by other actuators 11,12 as shown in FIG. 3b.

Another method for removing interstitial or body fluid out of the hole is to make very fine communication bores 13 into the thin lamella 1,2 as shown in FIG. 4. The skin cannot dome through these small holes but interstitial fluid can pass, for example by means of capillary action. This method has the advantage that the opening in the skin does not have to be exactly aligned in front of the opening in the lamella. A rotating hollow tube 14 with a applied suction vacuum can remove the liquid that penetrates through the bores 13. The capillary transport of the liquid can be enhanced by placing small grooves at the side of the lamella that is in contact with the skin.

It also an option to fix the location of the opening zone in the skin with the location of the aperture in the lamella by securing the lamella probe on the skin by means of a band or other method.

In one embodiment of the invention (shown in FIG. 5), it is envisaged that a structure 15 (such as a needle, conduit or tube) may be inserted in the cut opening 17 in the skin. The penetration depth of this structure 15 can be mechanically controlled for example by means of a coil and a magnet carriage structure 19 that has the lamella 1,2 as a reference, Other methods, such as arrangements used in optical recording technology, can be used for controlling the depth of the structure.

The present invention has the advantage over techniques in which the end of a needle or other conduit is used to puncture or incise through the skin, in that by forming the cut or hole 17 separately, the delivery or sampling structure 15 that penetrates through the cut or hole 17 can be blunt or of plastic or other material, which will give less problems with damaging the skin, or blockage of the end of the structure 15. Also, the structure 15 can be of a different size compared to the cut or hole 17. In this way fluid can be more easily withdrawn or injected through the skin. The method also has the advantage that possible impurities on the skin are, due to the parallel component of motion to the skin of the skin removing means, first removed from the area where the skin barrier is opened by the skin removing means. Further, the system as drawn in FIG. 5 can be equipped with a force transducer 20 and means for moving the lamella, not shown. When the aperture 3 in the lamella 1,2 is not aligned with the opening 17 in the skin, the force on the hollow structure measured by the transducer 20 will be much larger and an adjustment movement of the lamella probe will be made until as the position of the opening 17 is aligned with the lamella probe. In this way the invention provides an automated system for finding the holes in the skin. This facility can be seen by comparing the system configurations in FIGS. 5 and 6.

The sampled interstitial fluid can be analysed to detect the interesting molecules such as glucose, lactate, etc., or even combinations of several different molecules. Further it is also possible to directly detect DNA. This analysis can be done in an analyser on board a combination device which also carries or incorporates the lamella probe. A wearable device may be provided having on board, the lamella probe and also possibly an analyser and possibly, additionally or alternatively, a substance delivery arrangement (such as a medicament delivery arrangement). Alternatively the analysis may be carried out remotely.

The invention has so far primarily been described in relation to substance sampling, however there are also important aspects in relation to delivery of substances (drugs etc) in a like transdermal manner.

There are several methods to apply substances, typically in fluid form, through the cuts or holes made in the skin. This can be achieved in a similar manner as withdrawal of the interstitial liquid, previously described. A similar hollow tube can be used for withdrawal and injection of fluid, but also separate tubes can be used. Additionally, other methods can be used such as using a piston 23 to press fluid from a reservoir 24 through a delivery aperture 25 co-aligned with the cut or hole in the skin 17. Also applying the liquid in a very well controlled way with techniques similar to those used for ink-jet printing may be utilised. With all methods it is possible, and often desirable, to control the fluid flow rate of drugs in accordance with to a special delivery program.

In a certain embodiment of the invention, the apparatus comprises a device in which sampling of interstitial fluid and delivery of a substance (typically in fluid form) into the body, can be combined. In one embodiment, several cut holes in the skin can be used for sampling of the interstitial liquid. The outcome of this measurement can generate a decision to deliver a drug. The doses of the drug can be adjusted to the appropriate level and be applied with the previously discussed methods. This method can also be made in a wearable device by means of a very small motor with a battery.

Another method is to make a rotating system with a combination of functions as shown in plan view in FIG. 8. The carousel carries a disinfecting station (1) enabling application of a disinfecting liquid or anti microbial agent; a cutting station (2) enabling the formation of a cut hole in the skin by the previously discussed method of doming the skin. A sampling station (3) where the interstitial liquid can be withdrawn and analysed (3). Based on the outcome of this result the appropriate drug doses can be applied from an application delivery station (4). The same position can be used for all four functions. However, also different positions on the skin can be used. Further, extra functions can be added, for instance a substance for promotion of faster healing of the skin after use. Also it is possible to make a disposable cartridge that can be used only once. This cartridge can be combined with a driving unit for withdrawal of the body fluid and injection of the liquid in the disposable cartridge.

An exemplary device for use in accordance with the invention to obtain and analyse interstitial fluid is shown in FIGS. 9a to 9c. In the arrangement shown the probe 111 includes a lamella foil 101 at a distal end, including a plurality of apertures 103. A rotary blade arrangement 107 includes a plurality of blades for slicing off a layer of skin domed by and projecting through apertures 103 in the lamella foil 101.

In operation, interstitial fluid is projected radially outwardly from the blade arrangement and captured in a circumferential absorbent test strip 155. The probe includes an analyser platform 165 for analysis of the interstitial fluid captured in the test strip 155. It is possible to replace the test strip 155 before each measurement, but is also possible to store test strip-material for more than one measurement. The analyser can be used to measure if sufficient skin has been removed. The interstitial fluid can be analysed by a variety of techniques including but not limited to chemical reaction, biosensors, physical measurement using light transmission, reflectance or emission, measurement of electrical properties of the fluid. As a result of this analysis it is possible to calculate the amount of drug which needs to be delivered to a patient. The result may be displayed or communicated to a drug delivery device. The value may also be stored so that a history of values can be built up.

An alternative embodiment of an exemplary device in accordance with the invention is shown in FIGS. 10a to 10f. In this embodiment the device 211 is provided with a strap 270 for securing to the body of a user. The device carries a rotary cartridge 275 received in an aperture 276 in the main body housing 278 of the device. The cartridge is supported to rotate in the housing 278 adjacent a skin doming aperture 203 defined in skin contacting probe portion 201 of the device. The cartridge 275 includes segmentally spaced stations arranged to carry out various functions as the cartridge is rotated. In FIG. 10c a blade station 207 is centered in the aperture 203 and a blade arrangement acts to scrape cut the stratum corneum.

The cartridge then rotates to the position shown in FIG. 10d in which a measurement station 289 is centered in aperture 203 and acts to measure the skin doming or capacitance to determine that the required cut has been achieved. If the required cut has not been achieved the cartridge is rotated and readjusted to carry out a further scrape cut operation and subsequent measurement step. Once the required cut is measured as having been achieved the cartridge is rotated to the position shown in FIG. 10e in which sampled interstitial fluid is analysed by an analyser station 295 of the cartridge.

The interstitial fluid can be analysed by a variety of techniques including but not limited to chemical reaction, biosensors, physical measurement using light transmission, reflectance or emission, measurement of electrical properties of the fluid. As a result of this analysis it is possible to calculate the amount of drug which needs to be delivered to a patient. The value calculated in the analysis step can be used in dosing a drug to be administered in the next rotary position by means of the delivery/administering station 265. Dosing can be regulated by contact time, by sensing skin properties, by measuring the amount of drug and bring this into contact with the skin, and other means. An additional step can be provided after the administration step to enhance skin repair by means of a skin care cosmetic, a light treatment, a disinfecting step or another treatment which increases skin repair. Subsequently the device can be taken off or remain secured to the user.

In certain embodiments it is possible to sample indirectly the interstitial fluid or other target materials through the cut skin without physically obtaining a sample. This can be achieved via indirect means such as Raman spectroscopy, light absorption or reflectance techniques or the like. This enables rapid results to be obtained and these techniques are enhanced by being applied in accordance with the present invention.

In the embodiments described the devices in their entirety may be disposable, or component parts may be disposable. Also components may be removable for replacement in which the removed components may be refurbished for replacement, or alternatively disposed of. In the embodiment of FIG. 9 for example, the absorbent test strip 155 may be removed and disposed of following use and analysis, being replaced for subsequent use. Alternatively the entire sub assembly including the lamella foil 101 and analyser platform 165 may be disposable to be replaced by a like sub assembly.

Similarly, in the embodiment of FIG. 10, the rotary cartridge 275 can be removable and disposed of to be replaced with a fresh replacement cartridge. In such an embodiment the blade station 207, measurement station 289, analyser station 295 and delivery/administration 265 stations are all effectively disposable. In other envisaged embodiments some components or stations may be disposable, whereas others are configured not to be disposable.

It should be noted that the embodiments described illustrate the invention and should not be taken to limit the scope of the invention. In particular the skilled addressee will readily understand that features described independently and separately in respective embodiments may be combined within the spirit and scope of the invention. The words ‘comprises’ and ‘includes’ should not be construed as excluding the presence of other items, integers or features, nor ruling out the possibility of no other items, integers or features being present. Non specific references to steps such as substance analysis and dosing of medicament should in the broadest sense be understood as not limited to being carried out specifically on board a sampling device nor specifically remotely, but construed as widely as possible as encompassing both situations.

1. An apparatus for substance sampling and/or substance delivery via the skin barrier, the apparatus comprising means for doming and removing skin.

2. An apparatus according to claim 1, wherein the apparatus comprises contact means for contacting the skin, and including doming means for doming the skin; and a skin removal arrangement arranged to pass adjacent and remove a layer of domed skin adjacent the doming means.

3. An apparatus according to claim 2, wherein the doming means comprises an aperture, the domed skin projecting through the aperture to be removed by means of the skin removal arrangement passing across the adjacent aperture.

4. An apparatus according to claim 3, wherein substance sampling and/or substance delivery is conducted via the dome forming aperture in the apparatus.

5. An apparatus according to claim 2, wherein the skin removal arrangement includes a blade or scraper element that passes across the doming means in a slice plane that is generally substantially parallel to the surface of the skin outside the domed skin zone.

6. An apparatus according to claim 1, including:
i) capture means for capturing a sample of fluid passing from the skin removal zone of the skin; and/or
ii) assist means arranged to promote movement of the substance across the skin removal zone of the skin; and/or
iii) analyser means for analysing a sampled substance; and/or
iv) administration means for administration of a medicament via the opening in the skin; and/or
v) securing means for securing to the body or a body part of an individual.

7. An apparatus according to claim 1, wherein the apparatus further includes a delivery or extraction conduit arranged to be inserted to penetrate into the opening of the skin.

8. An apparatus according to claim 1, including a non-invasive sampling arrangement for non-invasive sampling via the opening in the skin barrier without sampling of interstitial fluid.

9. An apparatus according to claim 1, including:
i) capture means for storage of a captured sample, the capture means (or one or more components thereof) being removable from the apparatus and disposable, to be replaced like for like; and/or
ii) analyser means for analysing a sampled substance, he an analyser means (or one or more components thereof) being removable from the apparatus and disposable to be replaced like for like; and/or
iii) administration means for administration of a medicament, the administration means (or one or more components thereof) being removable from the apparatus and disposable to be replaced like for like.

10. A method for sampling and/or substance delivery via the skin barrier, the method comprising doming the skin in a controlled manner and operating a skin removal arrangement to remove domed skin so forming an opening in the skin barrier; and either sampling interstitial fluid or other substance present at the cut zone, and/or delivering a substance from externally of the skin through the cut zone.
